(19) United States
(12) Patent Application Publication
(10) Pub. No.: US 2019/0206036 A1
(43) Pub. Date: Jul. 4, 2019

(54) METHOD AND SYSTEM THAT USE SUPER FLICKER TO FACILITATE IMAGE COMPARISON

(71) Applicant: Al Analysis, Inc., Bellevue, WA (US)

(72) Inventors: Douglas Patriarche, Bellevue, WA (US); Julia Patriarche, Nepean, CA (US)

(73) Assignee: Al Analysis, Inc., Bellevue, WA (US)

(21) Appl. No.: 16/228,246

(22) Filed: Dec. 20, 2018

Related U.S. Application Data
(60) Provisional application No. 62/608,468, filed on Dec. 20, 2017.

(51) Int. Cl. G06T 5/50

(52) U.S. Cl. CPC G06T 5/50; A61B 5/055

(57) ABSTRACT

The current document is directed to methods and systems that overcome the image-comparison problems attendant with the human visual system by leveraging the motion-detection capabilities of the human visual system. Despite our visual system lacking automatic image-difference detection, our visual system does have an inherent ability to detect motion within our visual field and to direct our attention to this motion. This ability probably evolved as a result of the advantage provided by rapid detection of changes in our environment, including detection of the movement of predators or other threatening creatures, such as a snake in the bushes next to the campfire where we are eating. This method, usually referred to as “Flicker,” involves placing two images to be compared in the same location and rapidly alternating between them.

### CROSS-REFERENCE TO RELATED APPLICATION

This application claims the benefit of Provisional Application No. 62/608,468, filed Dec. 20, 2017.

### TECHNICAL FIELD

The current document is directed to medical imaging and, in particular, to methods and systems that rapidly alternate rendered medical images on a display device to facilitate identifying differences between the medical images by a human viewer.

### BACKGROUND

In general, the human visual system is poorly suited to the task of detecting differences between scenes and images. This is due to the fact that humans do not have a “hard wired” mechanism for comparing similar scenes or images. In fact, quite to the contrary, our visual systems are geared towards abstraction. When we see a tree, we see a tree, but we do not see: the exact position of every leaf, the exact shape and texture of the bark, the exact shade of green of the leaves, etc. Otherwise, our minds would be overwhelmed by useless detail—the presence of a tree in our midst is potentially important to us, but the exact position of every leaf on that tree, or the precise shade of green of the leaves, is generally not. Further, when the wind blows and the position of the leaves changes, it is useful for us to perceive the wind-blown tree as the same tree, and not a different tree, which is facilitated by abstracting the tree from the high-granularity details. Thus, our visual systems perceive the world in terms of abstractions, in order to prevent us from becoming overwhelmed by unneeded detail and to maintain continuity of perception. We are able to perceive the subtle details, for example, the exact position of a leaf, but only when we explicitly examine and attend to that detail. We can additionally perceive differences in subtle detail, but in general only if we intentionally attend to those details and explicitly compare them in our minds.

Our abstraction-based visual-system architecture has significant implications, including in fields of neuropsychology referred to as “change detection” and “change blindness.” In change detection, an individual first looks at a scene, then closes his or her eyes, looks away, etc., in order to induce a discontinuity, and finally looks at an identical or very similar scene (i.e. a scene that may or may not contain subtle differences from the first scene) to attempt to determine any differences between the first-viewed and later-viewed scene. An experimental method to explore this phenomenon may consist of
1) Directing a test subject view a scene, then directing the test subject close his or her eyes, while a small change is (or is not) made to the scene, and finally directing the test subject to open his or her eyes, view the (new) scene and determine the differences
2) Directing the test subject to view a photo on a computer screen, then directing the test subject close his or her eyes, while the photo on the computer screen is swapped for a photo that is nearly the same or entirely the same, and then directing the test subject to open his or her eyes, view the (new) photo, and determine the differences,
3) viewing two very similar or entirely the same photos side by side.
   The point of all three methods, above, is the discontinuity. The subject needs to close his or her eyes, look away, or even blink—this is sufficient to ‘flush his or her visual buffer’. A large number of studies in the neuropsychology literature have shown that this change detection task is very difficult; the time required to find differences using such a viewing mode is significant, and the likelihood of missing differences using such a viewing mode is also significant. In fact, there is a whole genre of games, in which the user attempts to accomplish this “difference detection” task.

Normally, when attempting this task (using the side-by-side viewing presentation), a user will look back and forth and back and forth between the two images, attending to one item and then the next and then the next, looking for changes, each time intentionally making a comparison. Rarely, an individual will attempt to “gestalt it” by studying one of the images, memorizing every item and detail of every item, then moving on to the other image, attending to each object and feature of the second image, and intentionally comparing each with the corresponding object and feature in the first image. The point is that there is no mechanism in the human visual system to automatically effect a comparison between two discontinuous images/scenes. In order to detect any differences, the viewer explicitly attends to every detail of the scene in both images, and then consciously make the comparison. Failing to do so dramatically increases the likelihood of failing to recognize the differences that are present, if any. The above approaches to the difference detection task are very time consuming and there is a significant likelihood of missing a change that is present.

There are real world situations in which the above discussed characteristics of our visual systems becomes a problem, however. One such situation is the comparison of astronomical images (for example photos of the night sky) to identify rapidly changing celestial bodies. In the earlier days of astronomy, photos of the night sky taken on different nights were compared in order to identify planets. Due to the fact that planets move rapidly in comparison with the “background” of much more distant stars, planets can be detected by establishing that they are in a different position of the night sky from one night to another (or even from one time of one night to a different time of the same night). However, comparing photographs of the night sky side-by-side in order to spot differences is time consuming and error prone.

Another real-world situation in which the above discussed characteristics our visual system are problematic is the comparison of serial medical imaging studies. Frequently, in medical practice, a given patient will have the same body part (for example, the brain) imaged periodically (for example every 3 months) to watch for recurrence of disease or to identify and characterize response to therapy. Currently, the standard practice is to view the images (for example, an MR scan obtained 3 months in the past and a current MR scan) side-by-side. The radiologist uses one of the above approaches (looking back and forth repeatedly, or first memorizing the previously obtained, or baseline, image by looking at each item in the baseline image, and then explicitly comparing the memorized details to the current, or follow-up, image by looking at each item in the follow-up image). As discussed above, this process is very time consuming and error prone.

### SUMMARY

The current document is directed to methods and systems that overcome the image-comparison problems attendant with the human visual system by (leveraging the motion-detection capabilities of the human visual system. Despite our visual system lacking automatic image-difference detection, our visual system does have an inherent ability to detect motion within our visual field and to direct our attention to this motion. This ability probably evolved as a result of the advantage provided by rapid detection of changes in our environment, including detection of the movement of predators or other threatening creatures, such as a snake in the bushes next to the campfire where we are eating. This strategy. usually referred to as “Flicker,” involves placing two images to be compared in the same location and rapidly alternating between them. “Flicker” provides for viewing a sequence of two or more images that are largely the same, and that may or may not contain differences with respect to one another, in order to facilitate a viewer's ability to quickly identify differences between the images by leveraging the human visual system's “motion detection” apparatus. In “flicker”, a system (e.g., a computer system) displays a rapidly alternating sequence of the two images, i.e. image 1, image 2, image 1, images 2, etc., as shown in FIG. 1.

### BRIEF DESCRIPTION OF THE DRAWINGS

FIG. 1 shows an alternating sequence of displayed images, in time.

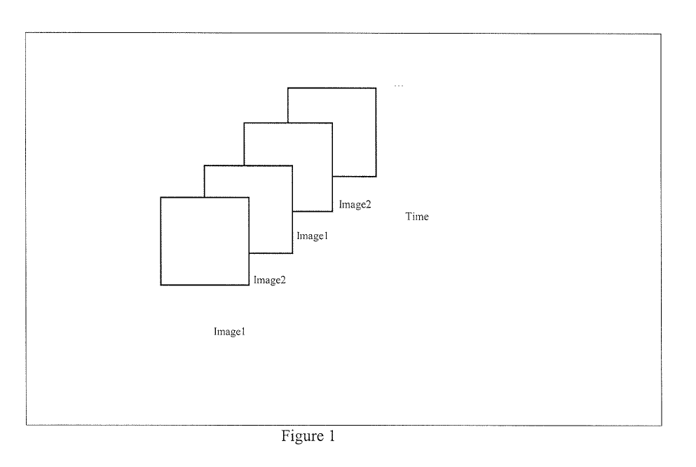

### DETAILED DESCRIPTION OF THE INVENTION

### The Basic Premise of Flicker

The flicker technique for detecting differences between images leverages the ability of the human visual system to detect motion—essentially discontinuities in the light intensity and/or color in a particular region of the viewer's visual field (which as mentioned above is essentially a ‘moving snake in the bushes’ detector). Any regions that are changing abruptly in time will tend to draw the attention of the viewer. Correspondingly, it is important to control the number of regions of an image that are changing abruptly. Thus, to compare two images using the flicker technique, those images must be spatially registered, in order to ensure that the subregion that is actually changing, occupies the same region of the viewer's visual field in both images and to ensure that regions that aren't actually changing don't appear as abrupt intensity changes due simply to misalignment of the images being compared.

### Super Flicker

In this disclosure, we describe a number of enhancements to the basic “Flicker” technique that we are designating “Super Flicker”. The basic premise of most of these enhancements is to increase the effectiveness of traditional “Flicker” by:
1) Suppressing abrupt transitions that are not relevant to the task at hand.
   The presence of regions of abrupt intensity transitions in an image that are not relevant to the difference detection purpose at hand waste the time of the viewer. Further, if there are an excessive number of such regions of abrupt intensity transition, the ability of such regions to draw the attention of the viewer will be impaired, and the user will be reduced to searching the image for regions of abrupt intensity transitions. Both of these situations increase the amount of time required to review the images and identify the differences.
2) Enhancing the abrupt transitions that are relevant to the task at hand, thus making such abrupt transitions more conspicuous to the viewer and more likely to draw the viewer's attention.
   We additionally describe one method to facilitate the practical deployment of flicker.

### Bias Field Correction

In some imaging modalities, images can suffer from “bias fields”; that is, regions where the image intensities have been scaled up or down in intensity due to the underlying image acquisition technology, lighting, etc., and not due to the actual item(s) being imaged. A simple example of this would be the sun moving across the sky during the day, altering which items in a scene are in shadow. If a pair of images, one from 8 AM, and one from 2 PM (on a sunny day), are compared using flicker, the changes in shadow would draw the attention of the user. However, if identifying changes to the actual objects is the objective, the shadow-related changes waste the viewer's time and might overwhelm the viewer's attention.

Similarly, magnetic resonance imaging suffers from a “bias field” problem, that results from a great many causes: heterogeneities in the main magnetic field, heterogeneities in the radiofrequency excitation, the specific geometry of the patient's body, and many other sources.

We have previously described one technique to address normalization issues but that additionally includes strategies for addressing such bias fields. Additionally, there are methods that are created solely to address more specific problems, such as bias fields in magnetic resonance images. One currently disclosed method is to view bias-field-corrected images in flicker mode, in order to suppress the “attention grabbing” effects of the abrupt local intensity changes in the viewer's visual field resulting from differences unrelated to the differences for which detection is desired.

### Intensity Normalization

In certain imaging modalities, the intensity characteristics of all of the objects in an image are altered from one image to the next. A simple example of this would be the situation in which the color or intensity of light has changed between two photographs of the same scene (for example comparing high noon images to sunset images, or sunset images to dusk images). In such a situation, when the two images are viewed in flicker mode, the entire scene will generate an abrupt discontinuity in the viewer's visual field, spoiling the attention-grabbing effect. The entire scene would be attempting to grab the viewer's attention simultaneously, and thus in effect no particular region would be distinctly grabbing the viewer's attention. Magnetic resonance imaging presents another example of an intensity normalization challenge. In magnetic resonance (MR) imaging, “acquisition parameters” can be changed, such that the same image type (e.g., T1) is acquired, but with somewhat different intensity characteristics. Even with the same acquisition parameters, the intensity normalization of MR images is generally not reproducible due to the vagaries of the scanners. Furthermore, in medical imaging, entirely different image types might be acquired of the same anatomical structure, containing similar information about that anatomical structure, for example: T1 MR, T2 MR, CT, etc. It might be desired to flicker between such images. However, in the situation in which intensity normalization as a whole has changed between one image and another in a flicker viewing mode, the entire image will produce an abrupt discontinuity in the viewer's visual field, and will attempt to draw the viewer's attention, thus spoiling the flicker mode's ability to draw the viewer's attention to a particular region of interest. A disclosed method is to flicker between intensity-normalized image volumes, rather than the original image volumes, so that a viewer is not distracted by changes resulting from different acquisition protocols or image types, and instead only sees changes due to the underlying biology.

\

### Color Enhancement of Changing Voxels

The above methods focus on reducing the abrupt intensity discontinuities that are not related to the task at hand. The “task at hand” might be detecting changes in serial magnetic resonance imaging studies, and intensity discontinuities not related to the task at hand might be changes in image intensities throughout the image due to modification of the acquisition parameters from one acquisition to the next. Another way to alter the flicker viewing mode so as to enhance its ability to direct a viewer's attention to the changes that are relevant to the task at hand (e.g., detecting biologically relevant changes in serial medical imaging studies) is to enhance the voxels that are detected by an artificial intelligence change-detection method as changing.

We have previously described automated computational methods for developing a map of the changes in a medical image. Such maps can be used to enhance original images that are then flickered, with the enhancements making the regions of actual/relevant changes more conspicuous and therefore more prone to draw the attention of the viewer.

One way to accomplish this is to alter the color characteristics of the voxels that the method thinks are changing, according to the degree of computed change, but without altering the intensity. For example, the voxels can be colored, and the saturation of the color can be used to represent the degree of change (for example, red may indicate that the voxels are changing, while pale pink may indicate slight changes, and scarlet may indicate dramatic changes). Alternatively, the hue can represent the degree of change. For example, cooler colors (e.g., pale blue) can be applied to mild changes and hot colors (e.g., scarlet) can be applied to more dramatic changes.

### Interleaving a Color Change Map

Another approach to enhancing the attention-grabbing capacity of flicker by using a change map derived from an artificial-intelligence method is to interleave the anatomical volume superimposed with the color change map with the two flickered volumes, so that the order of the flicker would be: image 1, image 2, image 2 with color map superimposed, image 1, image 2, image 2 with color map superimposed. It is important to show the color map superimposed on one of the two images being compared, and not just the color map with a black background, in order to avoid task irrelevant discontinuities in the black areas. Using this technique, the user can direct his or her attention not only to motion (i.e. regions of abrupt intensity discontinuity), but also to regions of color, making the process of identifying and comprehending changes easier.

### Application of an Intensity Scaling Change Map

Another way to apply an artificial-intelligence-derived change map in order to enhance the attention-grabbing capacity of changing regions is to use the artificial-intelligence-derived change map to enhance the abrupt intensity discontinuities of the regions that are detected by the artificial intelligence method as changing. If, for example, a region of a T2 brain MR is seen as normal in image 1, but T2 hyperintense (and abnormal) in image 2, and if the artificial intelligence method detected the change, the intensity of the region in image 2 can be increased, to enhance its capacity for grabbing the attention of the viewer. So, for example, if a region is normal in image 1, but is very slightly abnormal in image 2, the region that is judged to be becoming abnormal can be made even more T2 hyperintense in image 2 (for the sole purpose of increasing its attention-grabbing capacity).

Furthermore, on the flip side, the difference in intensity between image 1 and image 2 can be reduced in regions that have been judged to be unchanging by the artificial-intelligence-derived color change map. It would also be possible to scale the intensities of both image 1 and image 2 according to the artificial-intelligence-derived change map, so that voxels detected as unchanging by the artificial-intelligence-derived color change map are darker, while voxels detected as changing by artificial-intelligence-derived color map are brighter. This approach is similar to the approach, described above, of coloring voxels detected as changing by the artificial intelligence method (altering their hue or saturation), except that, in this case it would be the intensity that was being modified rather than the hue or saturation. In this case, the user can attune his or her visual search to look for “bright voxels” in addition to “flickering voxels”.

Flicker with Noise Reduction

Another feature of images that inevitably changes from one image (of a scene) to the next (image of the same scene) is sensor noise. It is possible to apply a smoothing filter to suppress some of this noise in both image 1 and image 2, so that changing noise between the images does not draw the viewer's attention and distract the viewer from task-relevant changes. Furthermore, the noise reduction process can be restricted to regions that have been judged to be unchanging by the artificial-intelligence-derived brain map.

### Flicker of Multiple Time Points

In some instances, there are more than two images to be compared. This is frequently the situation in radiology, for example, where a patient with multiple sclerosis or a brain tumor might be imaged using MR every 3 months for years. In such a situation, it is possible to generate the following flicker sequence: image 1, image 2, image 3, image 4, image 1, image 2, image 3, image 4, etc. One challenge with such an approach, is that flicker works by drawing the user's attention to dramatic intensity discontinuities in time. Creating a loop with too many images in sequence can reduce this effect, by making the changes appear smoother over time.

In order to mitigate this effect, the following approaches can be taken:
1) Select from the sequence of images, the pair of images possessing the greatest difference (according to artificial-intelligence-derived change maps) and flicker those instead.
2) For each region of change, select the pair of images in which the change in that region is most dramatic, and insert that pair of images next to each other in the flicker sequence.
3) Flicker images from the sequence in a random/changing order (which is basically a way to accomplish the previous approach (2), above, with much less computational complexity.

### Non-Linear Spatial Registration Prior to Flicker

Some structures that might be desirable to compare using flicker have the capacity to spatially deform nonlinearly between acquisitions. Two examples include inflation or deflation of the lung and bending of the elbow. If two images of lungs that were differently inflated in the two images are rigidly registered and flickered, virtually every region of the image appears to be changing, due to the difference in inflation. However, detection of difference in inflation is not typically task-relevant. Thus, non-linear spatial registration (i.e. “warping”) can be applied to the lungs, so that structures changing only due to normal biological function would be de-deformed, while structures changing due to an underlying disease process (e.g., the evolution of a tumor) are left alone. Thus, under flicker viewing mode, normal structures do not appear to change, while changing abnormal structures appear to change.

### Multiple Linear Spatial Registrations

Another approach to flickering such deformable structures is to derive multiple rigid body spatial registrations, so that, for each location in image 1, there exists a rigid body registration transformation that is “most optimal” for aligning that local neighborhood to the corresponding local neighborhood in image 2. The user can then select the region of interest by dragging the mouse pointer over a duplicate of image 1, while image 1 and image 2 flicker in another region of the screen, but with a rigid body registration applied to image 2 that results in the most appropriate alignment between image 1 and image 2 in that region.

This technique works extremely well for structures like the elbow, where there are two rigid subunits that change orientation relative to one another under normal conditions. It also works for structures, such as the lung, under conditions of inflation and deflation, although, in the case of the lung, there need to be a large number of rigid body transformations to cover the entire image area.

### Flicker for Detection of Changes Using Different Viewing Modes

In medical imaging and other applications, an “image” may consist of a 3D (or even 4D) volume of data, rather than a single 2D image (e.g., a photograph). There are a number of possible flicker viewing modes for 3D data:
1) Individual 2D slices through the volume may be flickered.
2) Arranging a set of 2D slices covering the entirety of the 3D volume (“tile mode”) and flicker all tiles at once. This increases the speed of review using flicker (rather than reviewing one slice, then the next, and then the next).
3) It is additionally possible to flicker a transparent 3D volume rendering of the 3D object, to permit the viewer to review the entire volume at once.
   Considering 4D data (for example 4D cardiac MR, where there are 3 spatial dimensions and one-time dimension), it is possible to review the data using flicker. The first step is to rigidly align the images both in time and space (rather than just in space, as is done with 3D data). Thus, at each point in time (and correspondingly at each point in the heart beat), there is a volume in acquisition 1 that is spatially rigidly registered to a volume in acquisition 2. Then, any of the above flicker viewing modes can be applied, perhaps tiled, so that all points in time can be displayed on the viewer's screen simultaneously. For example, a volume render of heart beat sequence 0, acquisition 0 can be flickered with heart beat sequence 0, acquisition 1 in one part of the screen, while a volume render of heart beat sequence 1, acquisition 0 can be flickered with heart beat sequence 1, acquisition 1 in another part of the screen, and so on, so that all points in time are flickered at the same time. Or, a full tiling of each heart beat sequence can be flickered in different points on the viewer's screen (however, this requires a large portion of the screen real-estate).

### Highly Automated Determination of Optimal Flicker Frequency

There is no unique value for the appropriate flicker frequency, across all users and situations. The appropriate flicker frequency can vary significantly from user to user, and from situation to situation (for example the level of alertness, fatigue, intoxication of the user including due to the use of prescription medication, and due to the task at hand). While it is possible to provide the user with a user-selectable range of flicker frequencies, it is also possible that they might forget to alter the frequency, and might therefore not benefit optimally from the use of flicker. Additionally, an excessively slow flicker frequency results in increased time required to review an entire case (which is undesirable).

One technique for “automatically” setting the flicker frequency is to present the user with a flickering image, starting with extremely high flicker frequency (so high that the flickering is not perceptible to any user). The user is asked to indicate when he or she perceives flickering, and then the system gradually reduces the flicker frequency until the user selection is made. Then, the user is asked to respond to a question that requires interpretation of the images (for example: “in which image—image 1 or image 2—is the lesion larger?”). The flicker frequency is again gradually decreased until the user is able to indicate that he or she is able to perceive the answer to the question. The two frequencies are then automatically used, in order to facilitate two different tasks: first, identifying changes, and second comprehending changes.

### Simplified Deployment of Flicker Data

Ideally, a dedicated software application is used to view flicker data. However, in practice, a user might want to review flicker data using a viewer that does not support flicker viewing mode. A means to accomplish this is to store the flicker data in either a movie file (with all slices displayed in every frame of the movie, i.e. ‘tiled mode’), or in transparent volume rendered mode. On first glance, it would appear that such a movie file would be extremely large; however, since the entire movie simply alternates between two different frames, the movie is massively compressible. Likewise, the flicker data can be stored in a 4D volume (i.e. the type of image volume used to store 4D cardiac MR data). Thus, using a typical viewer, the user can select the slice(s) of interest, and then play the slice through “time” (which turns the flicker mode on and off).

Although the present invention has been described in terms of particular embodiments, it is not intended that the invention be limited to these embodiments. Modification within the spirit of the invention will be apparent to those skilled in the art. For example, any of a variety of different implementations of the currently disclosed methods and systems can be obtained by varying any of many different design and implementation parameters, including modular organization, programming language, underlying operating system, control structures, data structures, and other such design and implementation parameters. It is appreciated that the previous description of the disclosed embodiments is provided to enable any person skilled in the art to make or use the present disclosure. Various modifications to these embodiments will be readily apparent to those skilled in the art, and the generic principles defined herein may be applied to other embodiments without departing from the spirit or scope of the disclosure. Thus, the present disclosure is not intended to be limited to the embodiments shown herein but is to be accorded the widest scope consistent with the principles and novel features disclosed herein.

1. An image-comparison system that rapidly alternates two or more images to facilitate image comparison, with the images processed to decrease task-irrelevant differences and to enhance task-relevant differences.
